(19) United States
(12) Patent Application Publication
(10) Pub. No.: US 2026/0158203 A1
(43) Pub. Date: Jun. 11, 2026

(54) DEVICE FOR ENRICHING FLUIDS WITH ENRICHMENT GAS, USE, METHOD, AND ENRICHMENT APPARATUS

(71) Applicant: HBOX THERAPIES GMBH, Aachen (DE)

(72) Inventors: Niklas STEUER, Aachen (DE); Peter SCHLANSTEIN, Aachen (DE); Matthias MENNE, Hoorn (NL); Christiane HOFFMAN, Frankfurt (DE)

(21) Appl. No.: 18/708,634

(22) PCT Filed: Nov. 9, 2022
(86) PCT No.: PCT/DE2022/100830

§ 371 Date: Jul. 2, 2024

(30) Foreign Application Priority Data
Nov. 9, 2021 (DE) 10 2021 129 141.0

(51) Int. Cl. A61M 1/36; A61M 1/16

(52) U.S. Cl. CPC A61M 1/367; A61M 1/16; A61M 1/3603; A61M 2202/0007; A61M 2202/0014; A61M 2202/0021; A61M 2202/0208; A61M 2202/0413; A61M 2205/103; A61M 2205/3334; A61M 2205/50

(57) ABSTRACT

The invention relates to a device (1) for enriching fluids (F) with an enrichment gas in a high pressure region (2) within the device (1) when flowing through the device (1), wherein the high pressure region (2) extends between an inlet side (11) and an outlet side (12) and has a predefined length in the flow direction (D) of the fluid, comprising a pressure boosting unit (3), preferably a first peristaltic pump, at the beginning (21) of the high pressure region (2) for increasing a pressure of the fluid (F) relative to the ambient pressure (UD), a gas supply unit (4) in the high pressure region (2) for supplying the enrichment gas (AG) under increased pressure (HD2) relative to the ambient pressure (UD) to the fluid already under increased pressure (HD1), and a pressure reduction unit (5), preferably a second peristaltic pump, at the end (22) of the high pressure region (2) for reducing the pressure of the fluid (F) at least to an effective pressure (ND) and preferably for conveying the fluid (F) out of the device (1).

The invention relates to an apparatus for enriching fluids with an enrichment gas, wherein the enrichment gas is supplied to the already pressurised fluid under increased pressure relative to an ambient pressure. The invention further relates to a method for operating such an apparatus and to the use of the device in the case of blood as the fluid for extracorporeal carbon monoxide detoxification, for extracorporeal oxygenation of blood for ischaemia treatment or cancer treatment or support thereof, or for extracorporeal membrane oxygenation, and also to an enrichment apparatus.

The enrichment of fluids with gases is a well-known technique, for example to prepare fluids for subsequent processes or procedures. In addition to other fields of technology, this principle is particularly important in medical technology, as it may be used to prepare a patient's blood (fluid) in order to supply the patient with suitable blood for various different medical reasons. For example, carbon monoxide poisoning may be treated by at least partially removing carbon monoxide from blood enriched with carbon monoxide through the addition of oxygen. Nowadays, such patients are ventilated with oxygen or placed in a pressurised chamber with an oxygen environment. It is also beneficial to enrich the patient's blood with oxygen for other purposes such as ischaemia treatment, cancer treatment, support for cancer treatment, or extracorporeal membrane oxygenation (ECMO). In this, the oxygenation of the blood takes place extracorporeally in a suitable device.

In the article “Hyperbaric phototherapy augments blood carbon monoxide removal” by A. Fischbach etal, Lasers Surg. Med. 2021, pages 1-7, a device for the treatment of carbon monoxide poisoning is described in which oxygen at elevated pressure is added to the blood at normal pressure via a photo-ECMO device. The more the oxygen pressure exceeds the blood pressure, the greater the carbon monoxide elimination. In addition, the effect on carbon monoxide elimination oxygen uptake of illuminating the reaction chamber with light of a specific wavelength is being investigated.

It is desirable to absorb as much oxygen as possible into the blood as quickly as possible in order to shorten the blood processing time and thus the patient's treatment time.

However, increasing the pressure above a threshold pressure would lead to the formation of oxygen bubbles in the blood, which could trigger embolisms in the patient, for example. Exceeding a gas pressure on the gas side relative to the blood pressure would lead to outgassing of the membranes. In addition, depressurising the blood, which is supersaturated compared to the environment (patient), would lead to the formation of gas bubbles.

It would therefore be desirable to have a device by means of which the enrichment of a fluid with gas can be carried out quickly and effectively, but without the potential for the procedure carried out by means of the device to cause damage to the fluid or to the patient.

The object of the present invention is to provide an alternative or enrichment to the prior art.

According to a first aspect of the present invention, the object is achieved by a device (1) for enriching fluids (F) with an enrichment gas in a high pressure region (2) within the device (1) when flowing through the device (1), the high pressure region (2) being present between an inlet side (11) and an outlet side (12) and extending in the flow direction (D) of the fluid (F), comprising a pressure boosting unit (3), preferably a first peristaltic pump, at the beginning (21) of the high pressure region (2) for increasing a pressure of the fluid (F) relative to the ambient pressure (UD), a gas supply unit (4) in the high pressure region (2) for supplying the enrichment gas (AG) under increased pressure (HD2) relative to the ambient pressure (UD) to the fluid (F) already under increased pressure (HD1), and a pressure reduction unit (5), preferably a second peristaltic pump, at the end (22) of the high pressure region (2) for depressurising the fluid (F) at least to an effective pressure (ND) and preferably for conveying the fluid (F) out of the device (1).

The following related terms are explained here:

Insofar as indefinite articles and numerical indications such as “one”, “two”, etc. are used in the context of the present patent application, these are always to be understood as “at least” indications, i.e. as “at least one”, “at least two”, etc., unless it is clear from the context or the technical knowledge that only “exactly one”, “exactly two”, etc. is or may be meant there.

The term “fluid” refers to any type of fluid or liquid that can be enriched with an enrichment gas. In some embodiments, the fluid may be a physiological fluid, such as blood or blood plasma.

The term “high pressure region” refers to the region in the device in which the pressure on the fluid is greater than the ambient pressure of the device.

The high pressure region is made possible in the present invention, for example, by the presence of the pressure reduction unit as an additional flow resistor. By utilising the pressure reduction unit in the fluid system, it is thus possible to raise the pressure in the high pressure region above the level that would be necessary to simply overcome the flow resistance of the individual components in the high pressure region. This makes it possible to adjust the fluid flow and the fluid pressure in the high pressure region independently of each other.

The ambient pressure may be the normal air pressure, for example, if the device is present in a room with normal air pressure. The high pressure region may be a single continuous segment or may comprise a plurality of separate or interconnected segments consisting of two parallel hoses connected via Y-connectors, for example.

The high pressure region may be configured in such a way that no hose segment in the high pressure region having relevant elasticity at prevailing pressure is present upstream or downstream of a component, and if more than one component is present in the high pressure region, that the connections between said components are not hose segments. If connections are present in the high pressure region, said connections may be tubes without relevant elasticity that can withstand the prevailing pressure, for example, or, if said connections are hose segments, said segments may be provided with sheaths for limiting in a defined manner the expansion of the hose segments due to the prevailing pressure, e.g. implemented as pressure-resistant hose sheaths.

Optionally, the high pressure region may have a predefined length.

The term “of predefined length” refers to a length dictated by the apparatus that cannot be changed during operation of the device. Said length is defined by the installation location of particular components in the device and ranges from the pressure boosting unit to the pressure reduction unit, which have a fixed position as components of the device, which determines the predefined length.

The term “pressure boosting unit” refers to any type of component that can be used to increase the pressure exerted on a fluid.

The pressure boosting unit may be a pump, for example a piston pump, a centrifugal pump, or a peristaltic pump, such as a roller pump or a linear peristaltic pump. This list is not exhaustive.

The main purpose of increasing the pressure is to generate a fluid flow through the high pressure region. In the present invention, the pressure must be increased in particular to such an extent that the flow resistance of the pressure reduction unit is overcome. Without the presence of the pressure reduction unit, the pressure level in the high pressure region would therefore only rise through the pressure boosting unit to such an extent that the flow resistances of the other components are overcome.

The term “pressure reduction unit” refers to any type of component that can be used to reduce the pressure exerted on a fluid.

The “pressure reduction unit”, for example, limits the high pressure region in the direction of the outlet. This component reduces the previously built-up pressure in a high pressure region. In particular, however, the pressure reduction unit presents an additional flow resistance that makes it possible to build up the high pressure region in the first place. In this respect, the pressure reduction unit also acts as an intentional pressure boosting unit for the high pressure region, whereby the fluid pressure in the high pressure region is increased beyond the pressure that would be necessary solely for the conveyance of the fluid against the resistances of the other components in the device according to the invention.

In the prior art, however, the pressure in all circuits is increased to such an extent that all resistances can be overcome at a particular flow, whereby flow and pressure cannot be set independently of each other, as any change in pressure also changes the flow. Said component may be, for example, a passive element such as a hose clamp, an orifice, or a diameter restriction; or an active element such as a pump (for example a piston pump, a centrifugal pump, or a peristaltic pump, such as a roller pump or a linear peristaltic pump), which transports against the direction of flow of the fluid (at a particular flow capacity in the direction of flow) or at a lower delivery speed than the pressure boosting unit in the direction of flow. The pressure reduction unit may also be designed as another rotating component through which the fluid flows, so that the pressure in the fluid can be reduced by transferring flow energy from the fluid to a rotation of the rotating component, such as an impeller. In order to influence the reduction in pressure, the tightness of the rotating component in the housing thereof or the leakage of the fluid past the rotating component or the rotational resistance of the rotating component can be set or defined. The pressure reduction unit may also be a rotating fibre bundle, for example.

The term “flow direction” refers to the direction of flow of the fluid from the inlet side to the outlet side. In the present invention, the flow direction is not reversed by the apparatus, so that the pressure boosting unit always conveys the fluid in the same direction, the flow direction.

The term “enrichment gas” fundamentally refers to all gases that can be transferred into the fluid. These comprise, for example, ozone, oxygen, or any gas mixtures having different concentrations of one or more gases in another carrier gas.

The term “effective pressure” refers to the pressure that is applied to the fluid downstream of the pressure reduction unit. Depending on the application, the effective pressure may correspond to the ambient pressure of the device, the normal pressure of the air, or another pressure level.

The term “gas supply unit” refers to the component in which the enrichment gas is supplied to the fluid. This can be done either by direct contact of the enrichment gas with the fluid or by indirect contact and transfer through a gas-permeable surface (membrane) from a gas side to a fluid side. The gas side is the part of the gas supply unit through which the fluid does not flow, but which is separated from the fluid, for example by membranes, i.e. the side on which the enrichment gas is applied for transfer into the fluid. The term “gas discharge unit” refers to the component in which a gas to be removed is extracted from the fluid. What has been said for the gas supply unit with regard to the gas side and fluid side also applies in principle to the gas discharge unit. Depending on the partial pressure of the gases present in the fluid and on the gas side, the gas supply unit for one gas component can cause the enrichment of this gas component in the fluid, as well as acting as a gas removal unit if another gas component in the fluid has a higher partial pressure than on the gas side of the gas supply unit. The other gas component would then pass from the fluid to the gas side and be transported away with the adjacent gas flow.

The term “gas to be removed” fundamentally refers to all gases that are to be removed from the fluid. Depending on the application, this comprises, for example, gases that are not desired for the device, harmful gases, hazardous gases, or excess gases, or any gas mixtures having different concentrations of one or more such gases in the fluid. In the exemplary case of a treatment of carbon monoxide poisoning by means of the device according to the invention, CO is a gas to be removed.

The invention relates to a device for treating fluids at pressures above ambient pressure. In the application of the device, a fluid, which includes both any liquid and any dispersion, is conveyed through a system in which there is at least one pressure region (high pressure region) in which the fluid is brought into contact with an enrichment gas and in which an increased physical pressure acts on the fluid in the high pressure region. There may be other regions in the device, non-pressurised regions, in which the fluid is exposed to a pressure in the region of the ambient pressure and in which the fluid can also be brought into contact with gas. In addition to the pressure boosting unit, gas supply unit and pressure reduction unit, the high pressure region may also comprise one or more of the following components in which the fluid can be present or through which the fluid can flow: hose segments, hose connectors, connectors, chambers, cavities, gas removal unit, heat supply unit, heat removal unit, etc. In addition to the one high pressure region, the device may also comprise other high pressure regions also connected to the inlet and outlet side. Said further high pressure regions may be disposed sequentially or parallel to each other, so that the fluid flows in parallel lines through the parallel high pressure regions of the device or flows in one line through the high pressure regions of the device disposed in series. The individual high pressure regions may exert the same increased pressure or differently increased pressures on the fluid relative to the ambient pressure. The fluid and/or enrichment gas may be fed through or into the device, in particular through or into the high pressure region, at a continuous or pulsatile or discontinuous flow or a combination of said flow types.

The device is used to heavily enrich the fluid with enrichment gas. In the case of blood as the fluid, the blood is enriched with enrichment gas, for example oxygen, above normal physiological saturation values.

The device may be configured such that the mechanical load on the fluid is kept as low as possible. When the device is applied to biological fluids such as blood in a medical application, for example, a low mechanical load on the blood results in minimal damage to the blood cells in the blood. The design of the device allows a low mechanical load on the fluid.

The device according to the invention thus makes it possible to enrich a fluid with gas quickly and effectively without the gas intake causing unacceptable damage to the fluid or a patient.

In one embodiment, the increased pressure of the enrichment gas is higher than the ambient pressure, but lower than the increased pressure of the fluid.

This allows the fluid to be enriched with a high quantity of enrichment gas without the enrichment gas forming bubbles in the fluid. By avoiding bubbles in the high pressure region, it is also possible to prevent larger gas bubbles from accumulating at certain points in the device in the high pressure region. This would pose a great danger to the patient when blood is used as a fluid.

In a further embodiment, at least the gas supply unit is configured such that, in the event of a fluctuating increased pressure of the fluid, the increased pressure of the enrichment gas follows the fluctuating increased pressure of the fluid accordingly.

When using a gas supply unit and/or a gas discharge unit in the high pressure region, the device according to the invention can be configured such that the pressure on the fluid side can be controlled and/or the pressure on the gas side can be controlled. Controlled in this sense means can be influenced and/or can be adjusted and/or can be measured and/or can be regulated and/or is defined by the construction or design. The gas supply unit and/or the gas discharge unit may, for example, be configured such that the controlled pressure on the fluid and the controlled pressure in the gas mean that the gas pressure is at a similar level to the fluid pressure in the high pressure region, but is always the same or slightly lower than the fluid pressure. If, for example, the gas pressure on the gas side is higher than on the fluid side, then a membrane present between the fluid and gas may be used for maintaining said pressure difference without gas passing from the gas side into the fluid side. For example, a direct mechanical or pneumatic coupling of the two pressures with each other can ensure this property, e.g. the gas pressure from the gas source can first act on a pressure-transmitting element on the fluid side and thus adjust the pressure in the fluid and then the same gas flow can run at a slightly reduced pressure through the gas side of the gas supply unit and/or the gas discharge unit. For example, the gas pressure could be regulated by means of the fluid pressure. In a further embodiment of the invention, the partial pressure of one or more gases dissolved in the fluid can be measured, e.g. by measuring the luminescence emission or with an electrochemical sensor. Depending on the measuring principle, the sensor may be present inside or outside the high pressure region. This information can then be used to regulate and/or control the gas pressure of the enrichment gas in the gas supply unit in such a way that certain limit values of the gas in the fluid are not exceeded. The limit values may, for example, be solubility limits of the gas in the fluid at the effective pressure, so that the gas cannot outgas from the fluid, or limit values to achieve a therapeutic benefit.

In a further embodiment, the increased pressure of the fluid in the high pressure region corresponds to an overpressure compared to the ambient pressure of 2 bar to 4 bar, preferably 2.5 bar to 3.5 bar, particularly preferably substantially 3.0 bar. In principle, it would be possible to operate the device at even higher pressure.

In the case of the pressure boosting unit and/or the pressure reduction unit implemented as first and/or second peristaltic pumps, the first and/or second peristaltic pump each has a hose segment,
in which its inner diameter tapers in one direction of the hose segment, preferably the inner diameter does not taper symmetrically, and/or
whose elasticity varies over the hose segment in such a way that expansion of certain segments can be prevented or at least reduced.

In a further embodiment of the first and/or second peristaltic pump designed as a roller pump, the rollers, their discharge surfaces and/or dimensions of a pump head are designed differently to increase or reduce the pressure.

In a further embodiment, in the first and/or second peristaltic pump, the hose segment is routed more than once in the same direction through the first and/or second peristaltic pump.

As a result, the pressure is built up in several passes through the peristaltic pump, which leads to a lower mechanical load on the fluid.

In a further embodiment, the second peristaltic pump is operated at a lower speed than the first peristaltic pump.

On the one hand, this builds up the high pressure region and, on the other hand, a pressure reduction from the increased pressure in the high pressure region to the effective pressure in the flow direction downstream of the second peristaltic pump can be achieved.

In a further embodiment, at least the first peristaltic pump works with complete occlusion at operating pressure in the high pressure region.

Occlusion is the closure of the tube segment, which is routed through the peristaltic pump, by the pressurised means (e.g. rollers) moved along the tube segment from the outside in the peristaltic pump, by means of which the fluid is moved through the tube segment. This prevents a gap in the tubing segment of the peristaltic pump due to incomplete occlusion, through which the fluid can flow back in front of the first peristaltic pump. In such a gap, a large mechanical load would be exerted on the fluid by the movement of the pressurised means and in such a gap, the pressure difference between the inlet and the high pressure region would force fluid backwards through the gap and exert a large mechanical load on the fluid. With complete occlusion, this load is prevented by the fluid only being pushed in front of the pressure means in the hose segment but not pressed against the wall of the hose segment and by the fluid only flowing in the conveying direction, so that the increased pressure on the fluid is built up gently with the lowest possible load on the fluid. This is particularly important in the case of blood as the fluid, as significantly fewer blood cells and blood components are damaged in the pressure boosting unit.

In a further embodiment, the pressure reduction unit is operated with a pumping action against the flow direction, whereby the pumping action is dimensioned such that fluid can pass through the pressure reduction unit in the flow direction.

This maintains the increased pressure in the high pressure region, but ensures that fluid flows out of the high pressure region.

In a further embodiment, the pressure reduction unit is operated as a second peristaltic pump with a sense of rotation against the direction of flow, whereby the second peristaltic pump is operated with only partial occlusion.

The partial occlusion allows fluid to flow through the second peristaltic pump in the direction of flow, although the second peristaltic pump pumps in the opposite direction.

In a further embodiment, the pressure boosting unit is operated as a first peristaltic pump and the pressure reduction unit is operated as a second peristaltic pump, wherein the first and second peristaltic pumps are designed as a common pump having a common pumping squeezing mechanism, wherein the first peristaltic pump is formed by a first hose segment inserted into the squeezing mechanism and the second peristaltic pump is formed by a second hose segment inserted along the first hose segment into the same squeezing mechanism.

This allows synergy to be achieved so that only one motor is required to operate the shared pump. This also allows the device to be built more compactly.

In a further embodiment, the first hose segment and the second hose segment are inserted into the common pump from the same side if the second peristaltic pump is to pump in the direction of flow. This is particularly advantageous because the motor has to apply force in one direction of rotation to operate the first peristaltic pump to build up pressure and has to apply a counteracting force in the opposite direction of rotation to operate the second peristaltic pump for controlled pressure reduction and in the present embodiment these two forces act in opposite directions and reduce the force requirement on the motor. Or the first hose segment and the second hose segment are inserted into the common pump from the opposite side if the second peristaltic pump is to pump against the direction of flow.

The embodiments of peristaltic pumps described below may be implemented individually or in combination in the first and/or second peristaltic pump:

(A) Hose segments with Diameter Changes:

When using a peristaltic pump at the beginning and/or end of the high pressure region, the peristaltic pump may be fitted with a hose segment that has a tapered hose segment in the region of the peristaltic pump. The tapered hose segment may consist of a hose having a continuous wall thickness and a tapered inner and outer diameter or of a hose having a variable wall thickness and a tapered inner diameter having a constant outer diameter or of a hose having a non-symmetrically variable wall thickness and a non-symmetrically tapered inner diameter. The tube segment in the region of the peristaltic pump may be round, oval, square, rectangular or any other shape in cross-section and the inner diameter and outer diameter in this sense denote the inner and outer lumen of a tube, even if it is not round in cross-section. In terms of the direction of flow of the fluid through the hose segment, the hose segment described may be configured such that it initially widens in one partial segment and narrows again in another partial segment, whereby the diameters before widening or after widening and before narrowing or after narrowing and in the event of possible repetition of such partial segments are independent of each other and do not necessarily have to be returned to an initial diameter. In order to achieve a pressure reduction at the end of the high pressure region, a hose segment having a widened diameter can be used.

### (B) Elasticity of the Hose Segments:

The first and/or second peristaltic pump may be fitted with a hose segment which has different material properties, e.g. has a lower elasticity at one end and/or on one side of the casing surface than at the other end and/or on the other side of the casing surface, so that, for example, an expansion of certain segments or sides of the hose segment can be influenced or prevented by, for example, increased internal pressure in the hose.

### (C) Dimensions of the Pumps and Hose Segments:

The first peristaltic pump at the beginning of the high pressure region may be fitted with a hose segment that has a different dimension than the hose segment with which the second peristaltic pump at the end of the high pressure region is fitted. The first peristaltic pump at the beginning of the high pressure region may also have different dimensions than the second peristaltic pump at the end of the high pressure region. When using a peristaltic pump that is designed as a roller pump, said pump may be designed differently in terms of the dimensions of the rollers and the pump head, both at the beginning and at the end of the high pressure region.

### (D) Two Hose Segments in One Pump:

The hose segment at the beginning of the high pressure region may be inserted into the same first peristaltic pump as the hose segment at the end of the high pressure region. The hose segments may have the same dimensions or different dimensions or at least one of the two hose segments may have the tapered hose segment described above and if both hose segments have tapered hose segments at the inlet and outlet of the high pressure region, said segments may be inserted into the same peristaltic pump in opposite directions, in the sense of the direction of flow of the fluid through the pressure region, one tapering and one widening, in the sense of the direction of flow of the fluid through the pressure region, or both tapering or both widening. If both hose segments are inserted into a peristaltic pump as a common pump, the hose segment at the beginning and end of the high pressure region may be designed so that said segments are in contact. The hose segments may contact each other at least in places and the hose walls may touch each other in such a way that the pressure and expansion in one hose segment influence the pressure and expansion in the other hose segment. For defined guidance of the two hose segments, said segments may be guided within the common pump, e.g. in contours or recesses in the rolling surface of a roller pump, in the rollers of a roller pump or in the base or plungers of a peristaltic pump. Said routing of the two hose segments is also possible in any other described version of a peristaltic pump in connection with the described system and is also possible with only one hose segment or more than two hose segments. The two hose segments in contact at the beginning and end of the high pressure region may also be configured such that a double-lumen hose is used, so that, for example, a higher pressure in the hose segment at the end of the high pressure region compared to the pressure at the beginning of the high pressure region leads to less filling of the hose segment at the beginning of the high pressure region, which in turn leads to a reduction in pressure in the high pressure region. Conversely, a lower pressure at the end of the high pressure region compared to the pressure at the beginning of the high pressure region can lead to more filling of the hose segment at the beginning of the high pressure region, which in turn leads to an increase in pressure in the high pressure region, so that, for example, an overall regulating system of the pressure in the high pressure region can be created. The lumens of the double-lumen tube may be disposed next to each other, inside each other, in different sizes or variable in shape and dimensions.

### (E) Hose Circulation and Multiple Hose Circulation:

When using a roller pump as a first and/or second peristaltic pump at the beginning and/or end of the high pressure region, the hose segments may be inserted into the respective roller pump at any angle >0°, especially not only in the region of 0-360°, but also beyond. This design may also be implemented when using a linear peristaltic pump, e.g. by placing the hose segment more than once in the same direction through the peristaltic pump. As a result, the pressure is built up in several passes through the peristaltic pump, which leads to a lower mechanical load on the fluid.

### (F) Number of Rollers and Hose Clamp Functions:

When using a roller pump as the first peristaltic pump at the start of the high pressure region, a roller may be used in the roller pump to build up pressure in the high pressure region and to convey the fluid, in which case a function that squeezes the hose segment may also be integrated at the outlet of the roller pump, for example. either synchronised with the rotational speed of the roller pump and actively opened and closed again or passively opened and closed so that the flow at the outlet of the roller pump is always actively released to the high pressure region when one roller has built up a certain pressure between the roller and the squeezing function point. When using a roller pump at the outlet of the high pressure region, a roller may be used in the roller pump to reduce the pressure in the high pressure region and to convey the fluid; a function that squeezes the hose segment may also be integrated at the inlet of the roller pump. When using a roller pump as the first and/or second peristaltic pump at the beginning and/or end of the high pressure region, roller pumps with two or three or more rollers may be used and the described squeezing function may also be integrated at the outlet of the roller pump at the inlet of the high pressure region and/or at the inlet of the roller pump at the outlet of the high pressure region.

### (G) Variable Roller Distance in Circumferential Direction:

When using a roller pump as a first and/or second peristaltic pump at the beginning and/or end of the high pressure region, the pump head may be configured such that the rollers do not always have the same distance to each other when rotating on the rolling surface of the roller pump in the circumferential direction of the pump head, but that one or more rollers move with or against the direction of rotation of the pump head at one or more defined points of the circulation or the hose segment, while one or more of the other rollers do not move with or against the direction of rotation of the pump head at the same time, so that the hose segment, which is blocked between two rollers by the squeezing by the rollers, e.g. becomes shorter or longer. For example, the hose segment, which is blocked between two rollers by the squeezing by the rollers, becomes shorter or longer and thus the pressure on the fluid in the hose segment increases or decreases. The property of a roller to change the distance in the direction of rotation to the other rollers at one or more specific points in the rotation may be realised, for example, with a spring force or pneumatic force accelerating or decelerating the roller in the direction of rotation of the pump head, whereby the accelerating or decelerating effect may act on the roller either from the outside, from a point not moving with the pump head, or may act on the roller from the pump head. A decelerating effect on one or more rollers at one or more points may be integrated, for example, by means of an elevation in the rolling surface (e.g. “lifting bump”) of the roller, which is only overcome after the pump head has continued to move and, for example, a spring force has been overcome. The property of a roller of at least short-term circulation displacement relative to the other rollers may also be implemented by a mechanism, such as a coupling or a gear connection, which between the pump head and the roller leads to an accelerating or decelerating movement of the roller with or against the direction of rotation of the pump head at one or more specific points of the circulation.

### (H) Contact Pressure of the Rollers or Plungers:

When using a roller pump or a linear peristaltic pump as the first and/or second peristaltic pump at the beginning and/or end of the high pressure region, the contact pressure, e.g. of the rollers on the rolling surface of the roller pump and the hose segment to be squeezed lying therein or e.g. of the plunger on the hose segment, may be applied by spring force or be dependent on the pressure in the high pressure region, e.g. by directly coupling the pressure in the high pressure region with the contact pressure. This can ensure a constant contact pressure of the rollers of a roller pump or the plungers of a linear peristaltic pump in order to compensate for effects such as thermal expansion of the hose segment or components of the peristaltic pump, manufacturing tolerances, wear effects of the hose segment or components of the peristaltic pump, material thicknesses in the case of a tapered or widening hose segment and so, for example, the hose segment can be squeezed or occluded only as much as necessary to seal the high pressure region. When using a roller pump as a first and/or second peristaltic pump at the beginning and/or end of the high pressure region, the rollers may be pressed against the roller pump's rolling surface and the hose segment to be squeezed therein using a spring force or pneumatic force. In this case, the rollers may also be mounted eccentrically, so that the rolling movement on the hose is not uniform, e.g. designed so that the pressure on the fluid in the hose segment, which is separated between two rollers by the pinches through the rollers, can be increased or decreased.

### (I) Shape of the Rollers and the Rolling Surface:

When using a roller pump as a first and/or second peristaltic pump at the beginning and/or end of the high pressure region, the rollers may have a non-cylindrical shape, such as a conical shape. On the one hand, the unwinding surface may be disposed parallel to the cone shape, for example, so that the hose to be squeezed is always compressed with the same distance between the roller and the unwinding surface. For example, when the hose segment, abutting the rolling surface, extends from the pump inlet to the pump outlet along the axial direction of the tapered roller toward the tapered end, the distance along the hose segment between two revolving tapered rollers becomes smaller, so that the pressure on the fluid in the hose segment separated between two rollers by the pinches through the rollers can be increased or decreased. On the other hand, the unwinding surface may be cylindrical, for example, so that the distance between the tapered rollers and the unwinding surface is not always the same and if, for example, the hose segment extends from the pump inlet to the pump outlet along the axial direction of the tapered rollers from the tapered end to the widened end, then the hose segment is squeezed more and more tightly so that a pressure build-up can occur. A similar effect may be achieved when cylindrical rollers roll along a conical rolling surface.

### (J) Eccentric Pump Head:

When using a roller pump as a first and/or second peristaltic pump at the beginning and/or end of the high pressure region, the pump head may be disposed eccentrically in relation to the rolling surface and the one or more rollers may be mounted on the pump head in such a way that they can be moved in the radial direction of the pump head. The rollers may, for example, be subjected to spring forces in the radial direction which apply the necessary squeezing of the hose segment and at the same time allow movement in the radial direction of the pump head so that, for example, the length of the hose segment squeezed between two rollers is shortened or extended when the pump head rotates and the pressure on the fluid enclosed in the hose segment is increased or decreased.

### (K) Externally Adjacent or Non-Adjacent Hose Segment:

When using a roller pump as a first and/or second peristaltic pump at the beginning and/or end of the high pressure region, the hose segment can be inserted in such a way that, for example, it is in contact with the rolling surface of the rollers everywhere or that it is not in contact with the rolling surface at any point, with the exception of the points where the rollers press the hose segment against the rolling surface of the roller pump. By suitably securing the hose segment at the inlet of the roller pump and/or at the outlet of the roller pump or within the roller pump, the hose segment may either be held in the described, fully attached position or in the described, fully non-attached position or it extends in a position between the two described positions. When using e.g. a roller pump at the beginning of the high pressure region, the hose segment may be inserted fully against the unwinding surface of the rollers. During operation, the hose segment may be filled from the inlet of the roller pump and when a roller engages with the hose at the inlet, pressure is built up towards the outlet of the roller pump and to prevent the hose from deforming at the outlet of the roller pump and, for example, kinking, the hose segment may be stiffened at this point, for example, so that no deformation occurs or the roller pump has, for example, a holder that guides the hose segment so that it does not deform. The ratio of the length of the hose segment within a roller pump when the hose is inserted in relation to the length when the hose segment is filled, e.g. either at the inlet of the high pressure region from the normal pressure region or e.g. at the outlet of the high pressure region from the high pressure region, may be used, for example, to increase or decrease the pressure on the fluid in the hose segment, which is separated between two rollers by the crimps through the rollers.

### (L) Multiple Roller Pumps or Pump Heads at the Beginning or End of the High Pressure Region:

When using a roller pump as a first and/or second peristaltic pump at the beginning and/or end of the high pressure region, a roller pump may consist of two or more roller pump units or consist of two or more pump heads or a pump head has two or more pump head levels disposed axially one above the other or it may consist of a combination of these variants, whereby the several roller pump units or pump heads or pump head levels and their rollers may have different sizes. By means of a suitable adjustment, the pressure on the fluid, which is separated between two rollers of the various roller pump units by the squeezing by the rollers, can be increased or reduced, for example, at the inlet of the high pressure region in a segment of the hose segment.

### (M) Passive Roller Pump at the Outlet of the High Pressure Region:

When using a roller pump as a second peristaltic pump at the end of the high pressure region, a passively rotating roller pump may be used, which is driven by the increased pressure in the high pressure region compared to the lower pressure in the normal pressure region downstream of the outlet of the roller pump and thus reduces the pressure on the fluid in the high pressure region to the effective pressure, for example normal pressure. The pump head can be adjusted, for example, with spring force or a magnetic resistance or a pneumatic force or a frictional resistance or a counteracting motorised unit such as an electric motor or stepper motor so that the rotational speed of the pump head leads to the desired flow through the hose segment with the desired pressure reduction.

### (N) Evacuated Pump Head:

When using a peristaltic pump as a first and/or second peristaltic pump at the beginning and/or end of the high pressure region, peristaltic pumps may be used in which an region between two squeezing bodies can be pressurised so that the external pressure on the hose segment in the pump can be controlled by incrementally and/or continuously increasing or reducing the pressure in this region and thus influencing the expansion of the hose. In the case of a roller pump, for example, the segment of the hose segment that is separated between two rollers by the crimping by the rollers may be designed to be tight to the environment so that this region can be pressurised and reduced with pneumatic pressure.

### (O) Drive of the Peristalsis

When driving a peristaltic pump as a first and/or second peristaltic pump at the beginning and/or end of the high pressure region, the peristaltic pump may be driven pneumatically or electronically or electrically or hydraulically. A roller pump may be rotated, for example, by a pneumatic drive, e.g. by a second hose segment inserted at the pump head, so that increased pressure in the second hose segment pushes peristaltic bodies such as rollers at the pump head so that the entire pump head rotates. If gas is used for the pneumatic drive, this may also be used after or before use as drive energy, e.g. to build up the pressure in the high pressure region and/or may be passed through a gas supply unit or a gas discharge unit, for example, in which gas is supplied to the fluid or gas is removed from the fluid. In a linear peristaltic pump, for example, the plungers of the pump may be driven pneumatically. If gas is used for the pneumatic drive, this may also be used after or before use as drive energy, e.g. to build up the pressure in the high pressure region and/or may be passed through a gas supply unit or a gas discharge unit, for example, in which gas is supplied to the fluid or gas is removed from the fluid.

In a further embodiment, the gas supply unit is designed as a membrane contactor supply unit.

Here, the membrane contactor supply unit may comprise a plurality of fluid-tight but gas-permeable membranes, whereby the membranes separate the fluid and the enrichment gas in such a way that the enrichment gas can enter the fluid via the membranes.

In a preferred embodiment, a first material stream of fluid or enrichment gas is passed through the membrane contactor supply unit, wherein the membranes in the first material stream are disposed in the membrane contactor supply unit, and wherein a second material stream of enrichment gas or fluid, which does not form the first material stream, is passed through the membranes separately from the first material stream.

Preferably, the fluid forms the first material flow and the enrichment gas the second material flow.

Enrichment gases may be present or consumed in the fluid in dissolved or bound or chemically converted form, whereby the forms of a gas present in the fluid are interdependent and also dependent on physical parameters such as temperature or pressure. The gas transfer from the gas side to the fluid side or vice versa may take place by diffusion or convective gas passage through a membrane. In an alternative direct gas-fluid contact, the gas transfer takes place at the interfaces between the enrichment gas and the fluid. If the enrichment gases are at least partially present in the fluid in dissolved form, the solubility may be expressed as a dissolved proportion of the gas concentration in the fluid or as a partial pressure. The partial pressure of an enrichment gas or other gases in the fluid may be influenced in the high pressure region either by the concentration of this gas on the gas side or by the quantity of gas supplied or by the gas pressure of the enrichment gas and the fluid pressure. If the concentration or partial pressure of a gas on the fluid side is higher than on the gas side, the gas will pass from the fluid to the gas side, e.g. in a gas discharge unit. If the concentration or partial pressure of a gas on the gas side is higher than on the fluid side, the enrichment gas will pass from the gas side to the fluid side, e.g. in a gas supply unit. If, for example, the fluid pressure is higher than the ambient pressure, the fluid can absorb more dissolved gas than at a pressure equal to the ambient pressure. The gas pressure, for example, may be used to influence the amount of enrichment gas transferred into the fluid or the amount of gas to be discharged from the fluid. If the gas pressure is, for example, above the ambient pressure and only slightly below the fluid pressure, a particularly high proportion of an enrichment gas can be transferred into the fluid in dissolved form or be present therein. If, for example, the gas pressure is below the fluid pressure or even below the ambient pressure, the dissolved proportion of a gas (e.g. a gas to be removed) in the fluid can be reduced. Normal pressure, positive pressure, negative pressure, or vacuum may be applied on the gas side. If, for example, negative pressure is applied on the gas side and an enrichment gas or gas to be removed is present in the fluid in such a large quantity that there is a lower concentration of this gas on the gas side in relation to the fluid side, the respective gas can be removed from the fluid in a gas discharge unit. When using the process with a system described here, the fluid pressures and gas pressures described may be varied and combined as required over the duration of the process. This means that different concentrations of all the different gases can be set in the fluid over the duration of the process. When using a peristaltic pump in the device, which may also be used for pressure increase and/or pressure reduction in the high pressure region, a negative pressure or vacuum can be generated on the gas side of the gas discharge unit, for example, by inserting a gas-carrying hose segment into the peristaltic pump, which creates a negative pressure or vacuum on the inlet side of the pump by conveying the gas.

In a further embodiment, the device therefore also comprises a gas discharge unit for discharging gas to be discharged from the fluid, disposed in the high pressure region downstream of the gas supply unit in the direction of flow.

Here, the gas discharge unit may be designed as a membrane actuator discharge unit.

If a gas (enrichment gas or other gases) is present in dissolved form in the fluid in the high pressure region and if this fluid undergoes a pressure reduction at the end of the high pressure region, the gas in the fluid may change to the gaseous state and bubbles may form in the fluid, both microscopic and macroscopic bubbles, for example. This effect of outgassing can be reduced or completely prevented, for example, by using a gas discharge unit in the high pressure region, which may be used both without a gas supply unit in the high pressure region and with a gas supply unit in the high pressure region. However, the position of the gas discharge unit in terms of the direction of flow of the fluid through the high pressure region may also be either upstream or downstream of the gas supply unit if this is used. If a gas removal unit is to fulfil the function of gas removal, it must have a lower concentration of a gas to be removed on the gas side than the concentration of this gas or the partial pressure of this gas on the fluid side. This concentration gradient may be influenced by the gas composition on the gas side or the quantity of gas supplied and discharged or by the pressure ratios in the fluid and in the gas. If the partial pressure of the gas to be discharged is higher on the fluid side than on the gas side, the gas is removed from the fluid by diffusion, for example. The gas side may be in a state of overpressure above normal pressure or in normal pressure or in a state of underpressure below normal pressure or in a vacuum. A gas removal unit may also be used to prevent outgassing when entering a low-pressure region by placing the gas removal unit upstream of the inlet to the low-pressure region to reduce the gas concentration. When using a gas discharge unit, a negative pressure or vacuum may be generated on the gas side by passing a gas flow through an element in which the Venturi effect prevails and that there is a connection point on the element at which a negative pressure is present with respect to the environment and that this connection point is connected to the gas side of the gas discharge unit. When using a gas supply unit, the gas flow for generating negative pressure or vacuum based on the Venturi effect may, for example, be partially or completely the same gas flow that goes into or comes out of the gas supply unit.

In a preferred embodiment, the fluid enriched with enrichment gas is passed through the membrane contactor discharge unit as a third material flow, wherein a plurality of fluid-tight but gas-permeable membranes are disposed in the third material flow so that the gas to be removed can pass from the fluid through the membranes and be discharged from the membrane contactor discharge unit.

Air with ambient pressure or negative pressure may be present on the other side (gas side) of the membranes, e.g. in the hollow fibre membranes. In the gas supply unit and/or the gas discharge unit, the membrane may be designed as a hollow fibre membrane or as a flat membrane. As an alternative to a membrane contactor, a compartment with a bubble column could also be used.

In a preferred embodiment, a vacuum pump unit is connected to the membranes for extracting the gas to be extracted from the fluid that has passed through the membranes. On the one hand, a gas to be discharged is a detrimental gas in the fluid, for example a toxic gas, or a gas that is harmless in itself, which is only present in the fluid in too high a proportion at increased pressure, so that it would bubble out of the fluid after the pressure reduction due to the lower external pressure.

In a further embodiment, the pressure reduction unit is designed as a gas discharge unit, preferably in that the gas discharge unit is designed for use as a pressure reduction unit by the arrangement and/or packing density of the membranes and/or flow routing of the fluid.

This is possible, for example, if the membranes are disposed in a suitable number, density and/or arrangement in relation to one another in the third material flow and/or the gas discharge unit as a pressure reduction unit has a suitable design, for example a suitable geometry, so that the friction of the fluid on the surfaces of the membranes and/or the gas discharge unit facing the fluid causes a pressure drop. In this case, for example, a second peristaltic pump would not be necessary.

The membrane may be a hollow fibre membrane or a flat membrane.

In a further embodiment, the gas supply unit and gas discharge unit are integrated in a common component.

Since gas supply and discharge lines are required in both components, the joint design in one component could utilise some components together, so that a reduction in the total number of components would be possible.

Here, for example, a further hose segment could also be inserted into the first or second peristaltic pump, which is connected to a gas outlet of the gas removal unit to provide a vacuum for improved gas removal from the fluid.

In a further embodiment, the gas supply unit and/or the gas discharge unit is combined with the pressure reduction unit.

The compartment containing the membrane with which the gas supply and/or gas removal is carried out is, for example, a cylindrical component that rotates. The fluid is supplied to this component radially from the outside and discharged radially from the inside. Due to the rotation of said component, the fluid experiences a rotational pressure or a centrifugal acceleration or a centrifugal force towards the outside, which causes a pressure build-up in said component radially outwards, so that a fluid passage from radially outside to radially inside is accompanied by a pressure reduction in the fluid due to the rotation of the component, while at the same time a gas supply or gas discharge takes place via the membrane contained in the compartment.

In a further embodiment, the pressure boosting unit and/or the pressure reduction unit is of a multi-stage design in order to achieve the desired increased pressure of the fluid in multiple steps starting from the pressure of the fluid on the inlet side or to reduce it to the effective pressure on the outlet side.

This means that the mechanical load on the fluid per pressure increase or pressure reduction step can be kept lower than would be the case with a single-stage pressure increase or reduction.

In a further embodiment, the fluid is a physiological liquid, preferably blood, and/or the enrichment gas comprises at least predominantly oxygen, preferably the enrichment gas has oxygen contents of greater than 80%, particularly preferably greater than 90%, even more preferably greater than 95%, or is pure oxygen.

The gas supply unit and/or gas discharge unit may be configured such that enrichment gas is supplied to the fluid via a gas supply unit, which may fulfil a function in the further course and whereabouts of the fluid. For example, a sufficient amount of enrichment gas may be dissolved into the fluid so that the type and quantities of the gas or gases are suitable for a mode of action with other substances or cells or gene-modifying substances in the fluid or support or prevent their mode of action. If one or more interfaces of the device are in contact with an external system, the device may be designed such that, for example, so much enrichment gas is introduced dissolved into the fluid that the type and quantities of the gas or gases cause gas components to be released from the fluid in the external system, e.g. in the form of microbubbles, and which have a functional benefit in the external system, such as visualisation with imaging methods of the e.g. microbubbles in the fluid flow.

If one or more interfaces of the device are in contact with an external system, a hydrostatic vacuum or suction pressure can act on the system and, if a gas supply unit and/or gas discharge unit is used, gas can pass through the membrane. To prevent this effect, the system described here may, for example, be configured such that a fluid pressure above the ambient pressure prevails in a gas supply unit and/or gas discharge unit present within a high pressure region, so that there is always a higher or equal pressure on the fluid side than on the gas side within a gas supply unit and/or gas discharge unit and no gas passage occurs, for example through a membrane from the gas side into the fluid side. The device may be configured such that no gas leakage occurs even if the system described here is located above the external system, e.g. spatially above a patient and thus above the patient's vascular system. This design of the device is particularly advantageous, for example, when the device is used in a clinical or non-clinical application in which the device is moved relative to the connected external system, such as the vascular system of a patient, and the device is sometimes below or at the same height or above the external system and the device is handled variably in its position and orientation in space.

The device may also be configured such that there is an interface to the outside at any point of the device, via which substances can be fed into the fluid and fluid can also be removed. When adding substances, it is possible, among other things, to add medicines or drugs or substances or markers for the visualisation of fluid components, either directly or by further visualisation methods, or substances that increase the absorption capacity of the fluid for gases, e.g. PFC fluids or substances that protect the fluid and or individual components of the fluid from thermal or chemical or mechanical influences and damage, such as substances that protect blood from cell damage or substances that influence the tonicity of the fluid, such as saline solution, so that either an isotonic or a hypotonic or a hypertonic fluid is provided.

Of course, the settings for the pressure boosting and pressure reduction units as well as for the gas supply and gas discharge units may be made manually, but this usually involves undesirable operating effort and would allow operating errors.

In a further embodiment, the device therefore comprises a control unit which is connected at least to the pressure boosting unit and the pressure reduction unit for controlling the increased pressure in the high pressure region by means of suitable data connections.

Preferably, the control unit is connected to the gas supply unit for controlling the supply of the enrichment gas and the gas pressure and/or to the gas discharge unit for controlling the discharge of the gas to be removed and the gas pressure.

The control unit preferably comprises one or more processors and one or more data memories for storing a control programme that is installed in the processor for execution for the operation of the device. Device parameters may be predefined in the control programme. However, the user could also enter device parameters into the device via an input interface so that the control unit controls the device according to the entered and/or stored parameters. The control unit makes it possible or easier for the user to operate the system and can provide him with information, e.g. measured values. The control unit may be partly mechanical, partly electrical, based on logic circuits or a combination of both. In this control unit, for example, a dependency of the gas-side pressure on the fluid-side pressure may be provided so that, for example, the gas-side pressure never falls below or exceeds the fluid-side pressure during operation of the system. The gas pressure in the gas supply unit may also be made dependent on the gas partial pressure in the fluid, for example, so that outgassing of the gas can be avoided when the pressure is reduced. Furthermore, the control unit may be used to synchronise the operation of several pumps depending on the system pressure, so that the user only has an influence on the fluid flow rate of the device, but the system pressure is always automatically maintained at a preset value, for example by the control unit adjusting the ratio of the speeds of the pumps or the opening of an orifice or the occlusion of a hose.

When a heat supply unit or heat dissipation unit is used in the device, it may, for example, consist of fibres or plates or other heat transfer surfaces which are supplied on one side with tempered water and on the other side with the fluid to which the gas enrichment process is applied. The heat supply unit or heat dissipation unit in the device may also be applied externally to the outer surface of hose segments, hose connectors, connectors, chambers and cavities. For example, the device may comprise a hose segment around which a sheath element is placed, which actively introduces or removes heat into the hose segment via the sheath. To maintain the temperature in the device, the system may be designed so that, for example, insulating material is placed around the components of the system, e.g. sheathing elements made of insulating material may be placed around a hose segment or a component may be placed in a housing made of insulating material. Temperature maintenance or temperature adjustment of the fluid may be used wherever the fluid leaving the outlet side of the device must have a specific temperature for the subsequent process. For example, blood as the fluid should have a temperature of approx. 37° C. before it is reintroduced into the patient.

For a low-pressure application of the device, only the components at the inlet and outlet would have to be replaced and the pressure-regulating units operated accordingly.

According to a second aspect of the present invention, the problem posed is solved by using the device described above, wherein blood is used as a fluid and oxygen is used as an enrichment gas, for extracorporeal blood treatment.

The blood treatment may be an extracorporeal carbon monoxide detoxification of the blood, an extracorporeal oxygenation of the blood for ischaemia treatment or cancer treatment or their support or an extracorporeal membrane oxygenation.

In an extracorporeal carbon monoxide detoxification of the blood as an embodiment of the above use, a patient with carbon monoxide poisoning is connected to the inlet and outlet side of the device via a catheter or double lumen catheter. The patient's blood flows into the device through the inlet side and through a hose segment and is conveyed into a high pressure region by the pressure boosting unit and then flows through a gas supply unit (as a detoxifier of the blood) and then through a gas discharge unit (as a degasser of the blood) and is conveyed out of the high pressure region by the pressure reduction unit. The blood may then also be returned to the patient's vascular system through a compliance and finally through a filter via the outlet side. The gas supply unit and the gas discharge unit contain, for example, fluid-tight and gas-permeable hollow fibre membranes. In the gas supply unit, for example, oxygen is supplied, passed on the gas side of the membranes, whereby oxygen is supplied to the blood under pressure and carbon monoxide is removed from the blood, and then removed again via a gas pressure reducer at a gas pressure in the gas supply unit that is, for example, just under 3 bar. In the gas removal unit, suction is applied to one side of the gas side of the membranes with a vacuum pump and the other side of the membranes is slightly open to the environment, so that some air is sucked in from the environment and the air is passed on the gas side of the membranes, all at a gas pressure slightly below ambient pressure, so that the oxygen previously introduced in dissolved form in the blood, which is still under increased pressure, is reduced again. Alternatively, no vacuum pump may be fitted to the gas side of the gas discharge unit and the gas side remains open to the environment. Depending on the surface region of the membranes used, this configuration may also be sufficient to reduce the dissolved oxygen.

Because the blood pressure in the gas supply unit is above the ambient pressure and the gas pressure is only slightly below the blood pressure, a very high quantity of dissolved oxygen can be supplied to the blood for extracorporeal oxygenation. The blood with a very high proportion of dissolved oxygen is then returned (perfused) to the patient's venous or arterial system via a return catheter after passing through the outlet side of the device. The return catheter can be located in larger vessels so that systemic perfusion of the blood takes place or in smaller vessels so that local perfusion of the blood takes place. In this way, ischaemic regions in organs such as the heart can be supplied with oxygen.

A method similar to that for treating ischaemic regions may be used with the device according to the invention in another embodiment of the above use to supply the hypoxic environment in a solid tumour with oxygen, so that the tumorous tissue has a higher concentration of oxygen. Hypoxic, tumorous tissue is less sensitive to radiotherapy, chemotherapy and immunotherapy, and oxygenation of this tissue increases the sensitivity of tumorous tissue to these forms of therapy. In addition, the body's own defence systems, e.g. T cells, are more active. The local introduction of blood with a high proportion of dissolved oxygen can support the efficiency of the forms of therapy mentioned, or the intensity of the radiotherapy, chemotherapeutic agent or immunotherapeutic agent may be reduced while still maintaining good efficacy. The introduction of an increased amount of oxygen into a tumour can also be beneficial for cancer treatment independently of radiotherapy or other treatment techniques.

A process similar to that for treating ischaemic regions may be used with the device according to the invention in a further embodiment of the above use in extracorporeal membrane oxygenation, even with a relatively small membrane region, to supply a blood flow in the low flow region (approx. to 500 ml/min-MiniECMO) with a lot of oxygen, so that the blood leaves the gas supply unit completely saturated with oxygen and additionally with a very high partial pressure of oxygen and thus this blood flow with a lot of oxygen can contribute to the emergency care of a patient with oxygen deficiency, so that in a very poor condition of the patient with critical oxygen deficiency, for example, invasive ventilation or extracorporeal membrane oxygenation (ECMO) with blood flows above 500 ml/min can still be dispensed with. Of course, the device may also be operated with higher blood flow rates (“normal” ECMO). For these purposes, the device may be designed so that the blood flows into the device from the patient's vascular system via the inlet cannula through the inlet side and is conveyed by the pressure boosting unit into a high pressure region, in which it flows through a gas supply unit and is then conveyed out of the high pressure region by the pressure reduction unit and then, after leaving the device, is returned to the patient's vascular system via the outlet side via a cannula. The high pressure region, which is limited by two roller pumps, for example, allows particularly high pressures to be generated in the blood and therefore a particularly large amount of oxygen to be introduced into the blood in the gas supply unit with a relatively small membrane surface region. Furthermore, the device may also comprise a gas removal unit, which may also be integrated with the gas supply unit in one component. The gas removal unit may be optimised in particular for the elimination of carbon dioxide, e.g. by means of short active hollow fibre membrane lengths, which continuously ensure low carbon dioxide concentrations on the gas side. The position of the gas discharge unit in the high pressure region is advantageous, as this supports carbon dioxide elimination due to the increased pressure. In an advantageous embodiment of the device, a negative pressure is present on the gas side of the gas discharge unit, but a normal or positive pressure is also possible.

In one embodiment, during extracorporeal membrane oxygenation, the device is operated with a blood flow through the device of less than 7 l/min, preferably less than 4 l/min, particularly preferably less than 2 l/min, even more preferably less than 1 l/min.

In addition to the above-mentioned uses, the device may also be used for organ preservation or organ transport.

According to a third aspect of the present invention, a method for operating a device described above for enriching fluids with an enrichment gas in a high pressure region within the device when flowing through the device, wherein the high pressure region extends between an inlet side and an outlet side with a predefined length in the flow direction of the fluid, comprising the following steps:
Introducing fluid into the device on the inlet side, preferably under ambient pressure;
Increasing a pressure of the fluid relative to the ambient pressure to an increased pressure with a pressure boosting unit, preferably a first peristaltic pump, at the beginning of the high pressure region;
Supply of the enrichment gas under increased pressure relative to the ambient pressure to the already pressurised fluid with a gas supply unit in the high pressure region in order to enrich the fluid with enrichment gas;
Depressurising the fluid to at least an effective pressure, preferably the ambient pressure, with a pressure reduction unit, preferably a second peristaltic pump, and Preferably conveying the fluid enriched with enrichment gas out of the device using the second peristaltic pump.

In one embodiment, the process comprises the further step of discharging gas to be removed from the fluid with a gas discharge unit disposed in the high pressure region in the flow direction downstream of the gas supply unit.

In one embodiment of the method, the gas to be discharged from the fluid, preferably with the gas discharge unit, is not completely discharged so that a desired amount of gas bubbles remain in the fluid after the fluid has been expanded in order to be able to visualise these bubbles in the fluid in downstream imaging methods for subsequent investigations of fluid dynamics.

Particularly in the case of blood as a fluid, the gas bubbles could be used to replace contrast agents which, according to current practice, are administered into the bloodstream of a patient in order to observe the fluid dynamics using imaging methods (e.g. Roentgen, MRI, CT) based on the temporal distribution of the contrast agents in the bloodstream, among other things. This may be used, for example, to detect circulatory disorders or vascular occlusions. This would also be possible with the detection of the intended bubbles. The size of the bubbles can be adjusted using the device. Preferably, the bubbles for this purpose have a very small diameter and therefore represent microbubbles in the blood that do not cause any damage to the patient, but can be observed in the blood using imaging techniques. If no gas discharge unit is provided for discharging the gas to be removed, the fluid would be supersaturated with respect to ambient pressure to such an extent that the enrichment gas would outgas during the pressure reduction or later in the patient by setting a corresponding enrichment gas pressure. This saves the unnecessary step of removing the gas.

In one embodiment of the process, the fluid for enrichment with the enrichment gas is conveyed several times through the device. For example, the fluid may be fed back into the high pressure region between the pressure boosting unit and the gas supply unit via a return line upstream of the pressure reduction unit. Alternatively, the fluid could also be channelled through a return line upstream of the outlet side to a point between the inlet side and the pressure boosting unit.

In one embodiment of the method, the step of introducing the fluid is preceded by pre-filling the device with a priming fluid and conveying it through the device in order to eliminate gases from the device with the priming fluid even before the fluid and the enrichment gas are supplied, preferably a saline solution is used as the priming fluid.

By pre-filling the device with a priming fluid, it is possible to ensure that there are no more bubbles in microscopic or macroscopic form later in the device for the fluid to be enriched, or that their number is at least uncritically low. For this purpose, the priming fluid for pre-filling the device is conveyed at least through the high pressure region in order to eliminate gases present or dissolved in the priming fluid there. The device may have one or more interfaces to the outside to a priming fluid reservoir and the priming fluid may be conveyed once or several times at least through a high pressure region. With the gas supply unit or an additional gas discharge unit, the device may be prefilled by applying a lower pressure on the gas side than the normal pressure. If one or more interfaces of the device are connected to the outside with a priming fluid reservoir, the priming fluid may be sucked into the device. The fluid-conducting device may be such that either a hose segment is pinched off by a component at the inlet of the high pressure region or a hose segment is pinched off by a component at the outlet of the high pressure region or there is no pinching off of the hose segments at the inlet and outlet of the high pressure region. The device may be prefilled with a priming fluid both during the manufacture of the device and when the device is used by a user. The priming fluid may be conveyed once or several times through at least one high pressure region or there is an interface to the outside where priming fluid may be supplied and/or removed. This interface may be present at one opening of the device or at two or more openings. If, for example, there are two openings as interfaces from the device to the environment, priming fluid may be supplied at one opening and discharged at the other opening. This interface may be designed so that, for example, the two openings are close together or are combined in one component and are physically connected, such as a double lumen tube or a double lumen catheter, or that these interfaces are not integrated in one component and are not physically connected to each other, such as two tube openings or two catheters. Another solution for eliminating gases is to convey the priming fluid through a low-pressure region in which outgassing is intentionally generated. The amount of gas now present in bubble form can then be eliminated, e.g. in a filter or a bubble trap. The priming fluid with a reduced gas load is then present at the outlet of the low-pressure region.

According to a fourth aspect of the invention, the object is achieved by an Enrichment apparatus,
in particular in the form of a device as described above,
comprising a high pressure region adapted to enrich a designated fluid with a designated enrichment gas in the high pressure region within the enrichment apparatus when flowing through the enrichment apparatus,
wherein the enrichment apparatus has an inlet side and an outlet side, and wherein the enrichment apparatus has a designated flow direction for the designated fluid extending from the inlet side to the outlet side,
wherein the high pressure region is present between the inlet side and the outlet side, wherein the high pressure region has a beginning and an end, wherein the beginning faces the inlet side and the end faces the outlet side,
whereby the high pressure region
   has a pressure boosting unit, in particular a pressure boosting pump, preferably a first peristaltic pump, at the beginning of the high pressure region, and
   has a gas supply unit in the high pressure region, wherein the gas supply unit has a supply cavity suitable for the enrichment gas, which is configured for supplying the enrichment gas under pressure, thus in operation into the fluid already under increased pressure,
wherein the gas supply unit has a flow resistance provider, in particular a valve,
whereby the flow resistance provider is present on the outlet side of the gas supply unit, in particular on the outlet side of the gas supply cavity,
wherein the flow resistance provider, in particular the valve, may have an adjustment option for a flow resistance, and
   has a pressure reduction unit, preferably a second peristaltic pump, at the end of the high pressure region, whereby the pressure reduction unit is designed as an additional flow resistance and for depressurizing the fluid and preferably for conveying the fluid out of the enrichment apparatus.

The embodiments listed above may be used individually or in any combination in deviation from the references in the claims in order to design the devices or methods according to the invention.

These and other aspects of the invention are shown in detail in the figures as follows.

FIG. 1: a schematic representation of an embodiment of the device according to the invention;

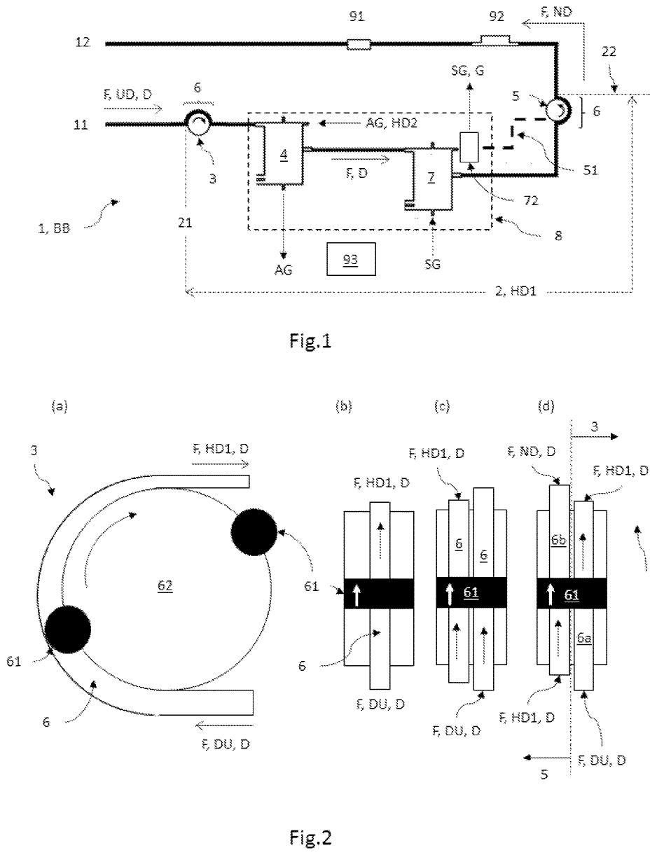

FIG. 2: a schematic representation of an embodiment of the peristaltic pump according to the invention as (a) first peristaltic pump in plan view, (b) first peristaltic pump according to FIG. 2a in side view; (c) first peristaltic pump in side view with double circumferential tube segment, and (b) first and second peristaltic pumps as a common pump in side view;

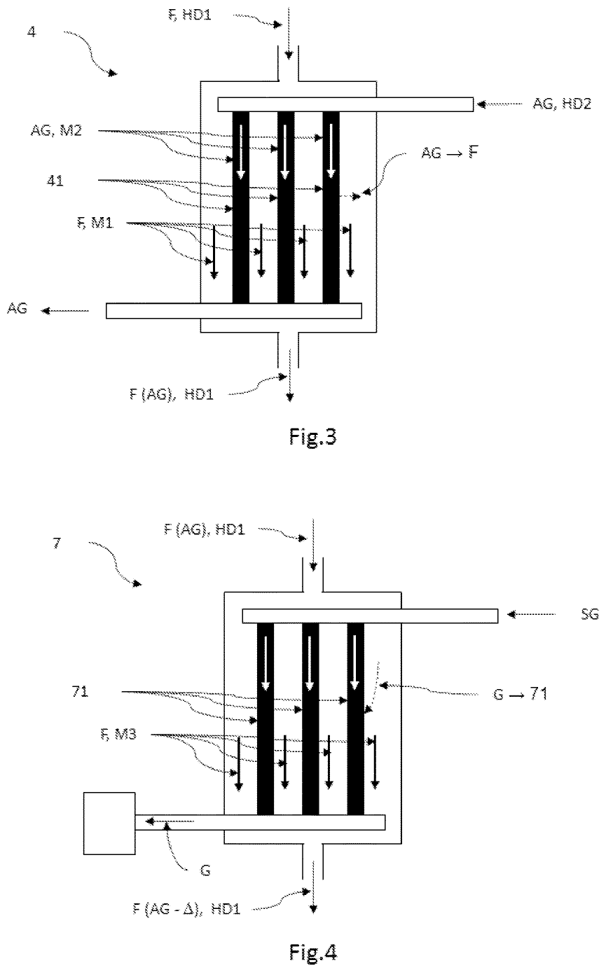

FIG. 3: a schematic representation of an embodiment of the gas supply unit according to the invention;

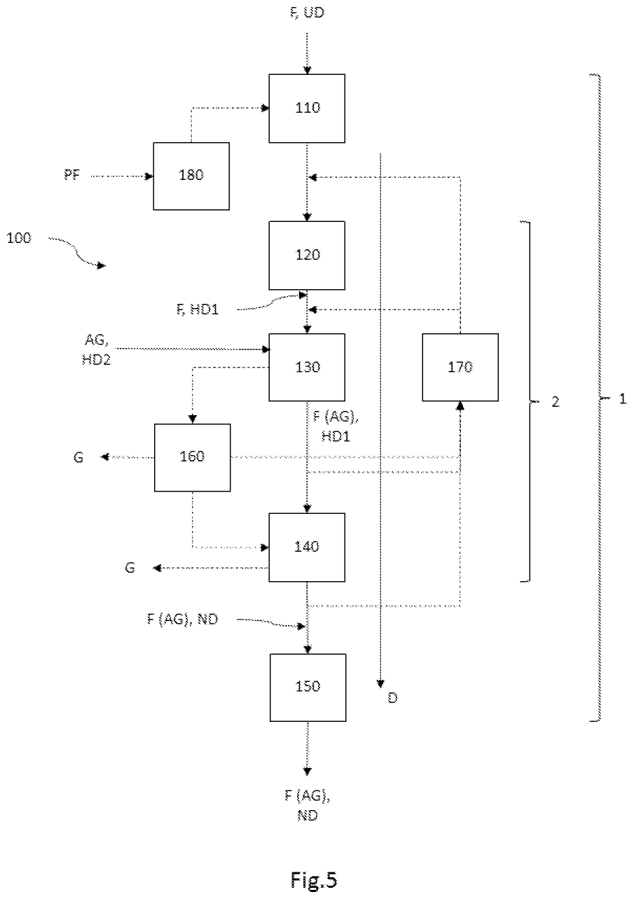

FIG. 4: a schematic representation of an embodiment of the gas removal unit according to the invention;

FIG. 5: a schematic representation of an embodiment of the method according to the invention; and

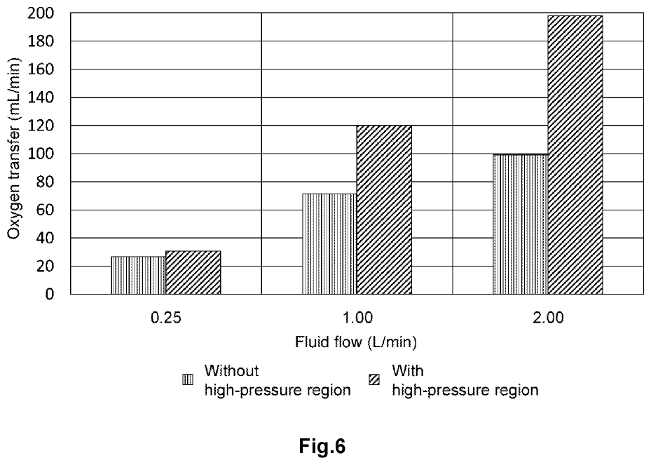

FIG. 6: Results from laboratory tests on the oxygen transfer rate with and without high pressure region at different fluid flows.

FIG. 1 shows a schematic representation of an embodiment of the device 1 according to the invention for enriching fluids F with an enrichment gas in a high pressure region 2 within the device 1 when flowing through the device 1. In the high pressure region 2, the fluid F has an increased pressure HD1 in the form of an overpressure compared to the ambient pressure UD of 2 bar to 4 bar, preferably 2.5 bar to 3.5 bar, particularly preferably substantially 3.0 bar. Here, the high pressure region 2 extends between an inlet side 11 and an outlet side 12 with a predefined length in the flow direction D of the fluid from a pressure boosting unit 3 to a pressure reduction unit 5 (see the region 2 delimited by dashed lines), with pressure-tight hoses or pipes connecting the components of the high pressure region 2. The pressure boosting unit 3 at the beginning 21 of the high pressure region 2 is intended to increase the pressure of the fluid F relative to the ambient pressure UD and is designed here as a first peristaltic pump. Furthermore, a gas supply unit 4 for supplying the enrichment gas AG under increased pressure HD 2 relative to the ambient pressure UD to the fluid already under increased pressure HD1 is disposed in the high pressure region 2. The gas supply unit is configured such that with a fluctuating increased pressure HD1 of the fluid F, the increased pressure HD1 of the enrichment gas AG follows the fluctuating increased pressure HD1 of the fluid F accordingly. In addition, a gas discharge unit 7 for discharging gas G to be discharged from the fluid F is disposed in the high pressure region 2 downstream of the gas supply unit 4 in the flow direction D. For this purpose, a purge gas SG flows through the gas discharge unit 7, taking with it the gas components that have passed from the fluid to the gas side and removing them from the gas discharge unit 7. The gas discharge unit 7 here also comprises a vacuum pump unit 72 for actively extracting the gas G to be discharged from the fluid F of the gas supply unit 4. It is indicated here that the pressure in the high pressure region 2 from the pressure boosting unit 3 to the pressure reduction unit 5 corresponds to the pressure HD 1 generated with the pressure boosting unit 3. This assumes that there are no pressure losses in the high pressure region 2. This is only ideally the case. For example, there may be a slight process-related pressure loss downstream of the gas supply unit 4 and/or downstream of the gas discharge unit 7. However, these pressure losses are small compared to the pressure increase achieved by the pressure boosting unit 3 and are therefore not explicitly taken into account in the drawings, so that the pressure specification HD 1 is shown for the ideal case of device 1 without pressure losses in high pressure region 2. The same applies to the FIGS. 3-5. The pressure reduction unit 5 at the end 22 of the high pressure region 2 is provided to reduce the pressure of the fluid F to at least an effective pressure ND and is designed here as a second peristaltic pump. The second peristaltic pump 5 can be operated here at a lower speed than the first peristaltic pump 3 or against the flow direction D in order to achieve a pressure reduction, whereby the pumping action is then dimensioned so that fluid can pass through the peristaltic pump 5 in the flow direction. For this purpose, the second peristaltic pump 5 is operated with only partial occlusion. The pressure reduction unit 5 can also cause the fluid F to be conveyed out of the device 1. The hose segment 6 in the first and second peristaltic pumps 3, 5 is explained in more detail in FIG. 2. As an alternative to the vacuum pump unit 72, a further hose segment 51 (shown dashed) may be inserted into the first or second peristaltic pump 3 or 5, which is connected to a gas outlet of the gas discharge unit 7. The pumping action of the first or second peristaltic pump 3 or 5 generates a negative pressure or a vacuum at the gas outlet of the gas removal unit 7 for the same purpose as the vacuum pump unit 72 instead of the vacuum pump unit 72. A filter unit 91 for removing unwanted components of the fluid and an elastic element 92 are disposed in series between the second peristaltic pump 5 and the outlet side 12. However, the reduction of the pulse of the second peristaltic pump 5 may also be realised differently than by the elastic element 92 or may be omitted. The increased pressure HD2 of the enrichment gas AG is higher than the ambient pressure UD, but lower than the increased pressure HD1 of the fluid F. In this embodiment, the gas supply unit 4 and gas discharge unit 7 are integrated in a common component 8, whereby the common component may be designed differently than shown here. The pressure boosting unit 3 and/or the pressure reduction unit 5 may be designed in multiple stages in order to achieve the desired increased pressure HD1 of the fluid F in several steps starting from the pressure of the fluid F at the inlet side 11 or to reduce it at least to the effective pressure ND at the outlet side 12. The fluid F may be a physiological fluid, preferably blood. The enrichment gas AG may comprise at least predominantly oxygen, preferably the enrichment gas AG has oxygen contents of more than 80%, particularly preferably more than 90%, even more preferably more than 95% or is pure oxygen. The device also comprises a control unit 93, which is connected to the pressure increasing unit 3 and the pressure reduction unit 5 for controlling the increased pressure in the high pressure region 2 by means of suitable data connections, and correspondingly also connected to the gas supply unit 4 for controlling a supply of the enrichment gas AG and/or to the gas discharge unit 7 for controlling the discharge of the gas G to be removed and/or for controlling the gas pressure as a function of the fluid pressure. For reasons of clarity, the data and control lines to the individual components are not explicitly shown. The device according to the invention may be used for extracorporeal blood treatment BB in the case of blood as fluid F and oxygen as enrichment gas AG. Such extracorporeal blood treatments are, for example, extracorporeal carbon monoxide detoxification of the blood, extracorporeal oxygenation of the blood for ischaemia treatment or cancer treatment or their support or extracorporeal membrane oxygenation. In extracorporeal membrane oxygenation BB, the device 1 can be operated with a blood flow through the device 1 of less than 7 l/min, preferably less than 4 l/min, particularly preferably less than 2 l/min, even more preferably less than 1 l/min.

FIG. 2 shows a schematic representation of an embodiment of the peristaltic pump 3, 5 according to the invention a as first peristaltic pump 3 in plan view, b first peristaltic pump 3 according to FIG. 2a in side view; c first peristaltic pump 3 in side view with double circumferential tube segment, and d first and second peristaltic pumps 3, 5 as a common pump in side view. The first and second peristaltic pumps 3, 5 each have a hose segment 6, 6a, 6b, in which the inner diameter of the hose segment 6, 6a, 6b tapers in the flow direction of the first peristaltic pump or increases in the second peristaltic pump, whereby the inner diameter may not taper or increase symmetrically. Its elasticity may also vary over the hose segment 6 in such a way that expansion of certain segments can be prevented or at least reduced. The first and/or second peristaltic pumps 3, 5 are designed as roller pumps, whereby the rollers 61, their discharge surfaces and/or the dimensions of a pump head 62 may be designed differently to increase the pressure. In the first and/or second peristaltic pump 3, 5, the hose segment 6 may be laid more than once in the same direction through the first and/or second peristaltic pump 3, 5, see FIG. 2 c for the example of the first peristaltic pump 3. In order to exert as little mechanical load as possible on the fluid, the first peristaltic pump 3 is operated here with complete occlusion at operating pressure, see FIG. 2a, where there is no gap between the rollers 61 and the wall of the hose segment 6, particularly under the prevailing pressure conditions. In FIG. 2d, first and second peristaltic pumps 3, 5 are designed as a common pump 3, 5 with a common pumping squeezing mechanism (rollers 61), wherein the first peristaltic pump 3 is formed by a first hose segment 6a inserted into the squeezing mechanism (rollers 61) and the second peristaltic pump 5 is formed by a second hose segment 6b inserted along the first hose segment 6a into the same squeezing mechanism (rollers 61). Here, the hose segment 6a of the first peristaltic pump 3 (right-hand side) tapers from the low-pressure side to the high pressure region 2. In the second peristaltic pump 5 (left side), the hose segment 6b also tapers from the side with low pressure to the high pressure region 2, only here the flow direction D is from the high pressure region 2 to the region with lower pressure, so that the first hose segment 6a and the second hose segment 6b are inserted into the common pump 3, 5 from the same side, since the second peristaltic pump 5 is to pump in the flow direction. The tapers are indicated by the larger/smaller hose diameters shown as differently sized hose protrusions above and below the roller pump. If the second peristaltic pump is to pump against the flow direction, the first hose segment 6a and the second hose segment 6b would have to be inserted into the common pump from the opposite side and the rollers 61 would have to be configured such that there is not complete occlusion, e.g. due to a step in the diameter of the roller 61.

FIG. 3 shows a schematic representation of an embodiment of the gas supply unit 4 according to the invention, which is designed here as a membrane contactor supply unit. The membrane contactor supply unit 4 comprises a plurality of fluid-tight but gas-permeable membranes 41, the membranes 41 separating the fluid F and the enrichment gas AG in such a way that the enrichment gas AG can enter the fluid F via the membranes 41. For this purpose, a first material stream M1 of fluid F is passed through the membrane contactor supply unit 4, the membranes 41 are disposed in the first material stream M1 in the membrane contactor supply unit 4, and a second material stream M2 of enrichment gas AG is passed through the membranes 41 separately from the first material stream M1. The membranes 41 may be hollow fibre membranes or flat membranes.

FIG. 4 shows a schematic representation of an embodiment of the gas removal unit 7 according to the invention designed as a membrane contactor removal unit. The fluid F enriched with enrichment gas AG is passed through the membrane contactor discharge unit 7 as a third material flow M3 and a plurality of fluid-tight but gas-permeable membranes 71 are disposed in the third material flow M3 so that the gas G to be removed from the fluid F can pass through the membranes 71 and be discharged from the membrane contactor discharge unit 7. In order to accelerate the degassing of the fluid F, a vacuum pump unit 72 is connected to the membranes 71 to extract the gas G to be removed from the fluid F that has passed through the membranes 71. If the membranes 71 are suitably designed and/or the membranes are suitably disposed and/or by suitable fluid routing and/or by suitable geometry/dimensions of the unit, the gas discharge unit 7 may also function as a pressure reduction unit 5 as a replacement for the second peristaltic pump 5 shown in FIG. 1. The membranes 71 may be hollow fibre membranes or flat membranes. In the case of an external flow, the membranes 71 may be tightly packed to reduce the pressure.

FIG. 5 shows a schematic representation of an embodiment of the method 100 according to the invention for operating a device 1 according to the invention for enriching fluids F with an enrichment gas AG in a high pressure region 2 within the device 1 when flowing through the device 1, wherein the high pressure region 2 extends between an inlet side 11 and an outlet side 12 with a predefined length in the flow direction D of the fluid F, comprising the following steps of introducing 110 fluid F into the device 1 at the inlet side 11, preferably under ambient pressure UD; of increasing 120 a pressure of the fluid F relative to the ambient pressure UD to an increased pressure HD1 with a pressure increasing unit 3, preferably a first peristaltic pump, at the beginning 21 of the high pressure region 2; of supplying 130 the enrichment gas AG under increased pressure HD2 relative to the ambient pressure UD to the fluid F already under increased pressure HD 2 with a gas supply unit 4 in the high pressure region 2 in order to enrich the fluid F with enrichment gas AG; depressurising 140 the fluid F to at least an effective pressure ND, preferably the ambient pressure D, with a pressure reduction unit 5, preferably a second peristaltic pump, and preferably conveying 150 the fluid F enriched with enrichment gas AG out of the device 1 with the second peristaltic pump 5. In this case, the further step of discharging 160 gas G to be discharged from the fluid F may also be carried out with a gas discharge unit 7 disposed in the high pressure region 2 in the flow direction D downstream of the gas supply unit 4. The removal 160 of the gas G to be removed from the fluid F with the gas removal unit 7 may not be complete, so that a desired amount of gas bubbles remain in the fluid F after the expansion 140 of the fluid F in order to be able to visualise these bubbles in downstream imaging procedures, for example in the patient, for subsequent investigations of fluid dynamics, vascular anatomy, etc. In one embodiment, the fluid F for enrichment with the enrichment gas AG may be conveyed several times through the device 1 or only through the high pressure region 2 170. In a further embodiment, the introduction 120 of the fluid is preceded by a pre-filling 180 of the device 1 with a priming fluid PF and its conveyance through the device 1 in order to eliminate gases G from the device 1 with the priming fluid PF even before the supply of the fluid F and the enrichment gas AG, preferably a saline solution is used as priming fluid PF. The priming fluid PF is passed through the entire device 1, usually several times in a circuit (not explicitly shown here).

FIG. 6 shows examples of results from laboratory tests on the oxygen transfer rate in millilitres per minute (ml/min) with devices with and without a high pressure region at different fluid flows in litres per minute (L/min). At a fluid flow of 0.25 l/min, the oxygen transfer rate in the device according to the invention with a high pressure region already exceeds the oxygen transfer rate in a device without such a high pressure region. If the fluid flow is increased to 1.00 L/min, the oxygen transfer rate increases for both devices with and without a high pressure region, whereby the difference in the oxygen transfer rate increases strongly in favour of the device according to the invention with a high pressure region. This effect increases significantly when the fluid flow is increased to 2.00 l/min. At this fluid flow, the oxygen transfer rate in the device according to the invention with a high pressure region is almost twice as high as in a device according to the state of the art. The high pressure region, which is limited by two roller pumps, for example, allows particularly high pressures to be generated in the blood and therefore a particularly large amount of oxygen to be introduced into the blood in the gas supply unit with a relatively small membrane surface region.

At this point, it should be explicitly pointed out that features of the solutions described above or in the claims and/or figures may also be combined, if necessary, in order to be able to implement or achieve the features, effects and advantages explained in a correspondingly cumulative manner.

It should be understood that the embodiment example described above is only a first embodiment of the present invention. In this respect, the embodiment of the invention is not limited to these examples.

### LIST OF REFERENCE NUMERALS

1 Device
11 Inlet side of the device
12 Outlet side of the device
2 High pressure region
21 Start of the high pressure region
22 End of the high pressure region
3 Pressure boosting unit, preferably a first peristaltic pump
4 Gas supply unit
41 Fluid-tight but gas-permeable membranes
5 Pressure reduction unit, preferably a second peristaltic pump
51 Further hose segment
6 Hose segment
6a First hose segment
6b Second hose segment
61 Rollers of the first and/or second peristaltic pump
62 Pump head of the first and/or second peristaltic pump
7 Gas discharge unit
71 Fluid-tight but gas-permeable membranes
72 Vacuum pump unit
8 Common component comprising gas supply unit and gas discharge unit
91 Filter unit
92 Elastic hose element
93 Control unit of the device
100 Method of operating the device according to the invention
110 Introducing fluid into the device on the inlet side
120 Increasing a pressure of the fluid relative to the ambient pressure to an increased pressure by means of a pressure boosting unit
130 Supplying the enrichment gas under increased pressure relative to the ambient pressure to the already pressurised fluid by means of a gas supply unit
140 Expanding the fluid to at least an effective pressure by means of a second peristaltic pump
150 Conveying the fluid enriched with enrichment gas out of the device by means of the second peristaltic pump
160 Removing gas to be discharged from the fluid by means of a gas discharge unit
170 Multiple conveying of the fluid through the device
180 Pre-filling the device with fluid and conveying the same through the device
AG Enrichment gas
BB Blood treatment
D Flow direction
F Fluid, for example a physiological fluid such as blood
G Gas to be discharged
SG Purging gas
HD1 Increased fluid pressure
HD2 Increased pressure of the enrichment gas
M1 First material flow
M2 Second material flow
M3 Third material flow
ND Effective pressure
PF Priming fluid
UD Ambient pressure

1. A device for enriching a fluid with an enrichment gas in a high pressure region within the device while the fluid flows through the device at a first increased pressure, the high pressure region being disposed between an inlet side and an outlet side and extending in a flow direction of the fluid the device comprising a pressure boosting unit disposed at a beginning of the high pressure region and configured to increase a pressure of the fluid relative to an ambient pressure a gas supply unit disposed in the high pressure region and configured to supply the enrichment gas at a second increased pressure relative to the ambient pressure to the fluid already under the first increased pressure, and a pressure reduction unit disposed at an end of the high pressure region and configured to depressurize the fluid to at least an effective pressure and to convey the fluid out of the device.

2. The device according to claim 1, wherein the second increased pressure of the enrichment gas is higher than the ambient pressure, but lower than the first increased pressure of the fluid.

3. The device according to claim 2, wherein the gas supply unit is configured to, when the first increased pressure of the fluid fluctuates, supply the enrichment gas at a fluctuating second increased pressure corresponding to the fluctuating first increased pressure of the fluid.

4. The device according to claim 1, wherein the first increased pressure of the fluid in the high pressure region corresponds to an overpressure compared to the ambient pressure of 2 bar to 4 bar.

5. The device according to claim 1, wherein the pressure boosting unit is a first peristaltic pump and the pressure reduction unit is a second peristaltic pump, at least one of the first or second peristaltic pump has a hose segment, and
wherein each hose segment includes at least one of an inner diameter which tapers in one direction of the hose segment or an elasticity which varies over the hose segment to reduce particular segments.

6. The device according to claim 5, wherein at least one of the first or second peristaltic pumps is a roller pump having a pump head and rollers each having a contact surface, the contact surfaces of the rollers being dimensioned differently from the pump head to increase or reduce pressure.

7. The device according to claim 5, wherein each hose segment is routed more than once in the same direction through the respective peristaltic pump.

8. The device according to claim 5, wherein the second peristaltic pump is operated at a lower speed than the first peristaltic pump.

9. The device according to claim 5, wherein at least the first peristaltic pump operates with complete occlusion at operating pressure.

10. The device according to claim 1, wherein the pressure reduction unit is operated with a pumping action against the flow direction, and wherein the pumping action is dimensioned such that fluid can pass through the pressure reduction unit in the flow direction.

11. The device according to claim 10, wherein the pressure reduction unit is operated as a second peristaltic pump having a sense of rotation against the flow direction, and wherein the pressure reduction unit is operated with only partial occlusion.

12. The device according to claim 1, wherein the pressure boosting unit is operated as a first peristaltic pump and the pressure reduction unit is operated as a second peristaltic pump, wherein the first and second peristaltic pumps are configured as a common pump having a common pumping squeezing mechanism, and wherein the first peristaltic pump is formed by a first hose segment inserted into the squeezing mechanism and the second peristaltic pump is formed by a second hose segment inserted along the first hose segment into the squeezing mechanism.

13. The device according to claim 12, wherein the first hose segment and the second hose segment are inserted into the common pump from the same side when the second peristaltic pump is to pump in the flow direction or the first hose segment and the second hose segment are inserted into the common pump from opposite sides when the second peristaltic pump is to pump against the flow direction.

14. The device according to claim 1, wherein the gas supply unit is configured as a membrane contactor supply unit.

15. The device according to claim 14, wherein the membrane contactor supply unit comprises a plurality of fluid-tight but gas-permeable membranes and wherein the membranes separate the fluid and the enrichment gas such that the enrichment gas can enter the fluid via the membranes.

16. The device according to claim 15, wherein, when a first material flow of the fluid or the enrichment gas is passed through the membrane contactor supply unit, the membranes in the first material flow are arranged in the membrane contactor supply unit, and a second material flow of the enrichment gas or the fluid is passed through the membranes separately from the first material flow, the first material flow being different from the second material flow.

17. The device according to claim 16, wherein the fluid forms the first material flow and the enrichment gas forms the second material flow.

18. The device according to claim 1, further comprising a gas discharge unit for discharging gas from the fluid and disposed in the high pressure region downstream of the gas supply unit in the flow direction.

19. The device according to claim 18, wherein the gas discharge unit is configured as a membrane contactor discharge unit.

20. The device according to claim 19, wherein the fluid enriched with enrichment gas is passed through the membrane contactor discharge unit as a material flow, and a plurality of fluid-tight but gas-permeable membranes are disposed in the material flow and gas-permeable membranes are disposed in the material flow such that the gas to be removed from the fluid can pass through the membranes and be discharged from the membrane contactor discharge unit.

21. The device according to claim 20, wherein a vacuum pump unit is connected to the membranes for extracting the gas to be removed from the fluid which has passed through the membranes.

22. The device according to claim 18, wherein the pressure reduction unit is configured as a gas discharge unit.

23. The device according to claim 15, wherein each membrane is a hollow fibre membrane or a flat membrane.

24. The device according to claim 18, wherein the gas supply unit and the gas discharge unit are integrated in a common component.

25. The device according to claim 18, wherein a hose segment is inserted into at least one of the pressure boosting unit or the pressure reduction unit and is connected to a gas outlet of the gas discharge unit.

26. The device according to claim 1, wherein at least one of the pressure boosting unit or the pressure reduction unit is of a multi stage design in order to achieve the desired increased pressure of the fluid in multiple steps starting from the pressure of the fluid on the inlet side or to reduce it to the effective pressure on the outlet side.

27. The device according to claim 1, wherein the fluid is a physiological liquid, and/or the enrichment gas comprises an oxygen content greater than 50%.

28. The device according to claim 1, further comprising a control unit connected at least to the pressure boosting unit and the pressure reduction unit for controlling the increased pressure in the high pressure region by means of suitable data connections.

29. The device according to claim 28, wherein the control unit is connected to the gas supply unit for controlling a supply of the enrichment gas and/or to the gas discharge unit for controlling the discharge of the gas to be removed and/or for controlling the gas pressure as a function of the fluid pressure.

30. The device according to claim 29, wherein the device is configured to measure a partial pressure of at least one dissolved in the fluid, and wherein the control unit is configured to regulate the gas pressure of the enrichment gas in the gas supply unit so that limit values of the at least one in the fluid are not exceeded.

31. A use of a device according to claim 1, wherein the fluid is blood and the enrichment gas is oxygen for extracorporeal blood treatment.

32. The use according to claim 31, wherein during extracorporeal membrane oxygenation the device is operated with a blood flow through the device of less than 7 l/min.

33. A method for operating a device for enriching a fluid with an enrichment gas in a high pressure region within the device when flowing through the device wherein the high pressure region extends between an inlet side and an outlet side of predefined length in a flow direction of the fluid the method comprising the following steps:
introducing the fluid into the device at the inlet side under ambient pressure;
increasing a pressure of the fluid relative to the ambient pressure to a first increased pressure with a pressure boosting unit at a beginning of the high pressure region;
supplying the enrichment gas under a second increased pressure relative to the ambient pressure to the fluid already under the first increased pressure with a gas supply unit in the high pressure region in order to enrich the fluid with the enrichment gas;
depressurising the fluid at least to an effective pressure with a pressure reduction unit; and
conveying the fluid enriched with the enrichment gas out of the device using the pressure reduction unit.

34. The method according to claim 33, further comprising the step of
discharging gas to be discharged from the fluid with a gas discharge unit disposed in the high pressure region downstream of the gas supply unit in the flow direction.

35. The method according to claim 34, wherein the gas to be discharged is not completely removed from the fluid so that an amount of gas bubbles remain in the fluid after depressurising the fluid such that the gas bubbles in the fluid may be visualized.

36. The method according to claim 33, wherein the fluid is conveyed through the device more than once.

37. The method according to claim 33, wherein a priming fluid is introduced into and conveyed through the device to eliminate gases from the device before the fluid is introduced into the device.

38. An enrichment apparatus, comprising a high pressure region configured to enrich a designated fluid with a designated enrichment gas in the high pressure region within the enrichment apparatus when the designated fluid is flowing through the enrichment apparatus,
wherein the enrichment apparatus has an inlet side and an outlet side, and wherein the enrichment apparatus has a designated flow direction for the designated fluid extending from the inlet side to the outlet side,
wherein the high pressure region is disposed between the inlet side and the outlet side, the high pressure region has a beginning and an end, and the beginning faces the inlet side and the end faces the outlet side,
wherein the high pressure region
has a pressure boosting pump disposed at the beginning of the high pressure region and
a gas supply unit with a supply cavity suitable for the enrichment gas, which is configured for supplying the enrichment gas under a first pressure into the fluid having a second pressure,
wherein the gas supply unit has a flow resistance provider, disposed on the outlet side of the gas supply unit
and a pressure reduction unit disposed at the end of the high pressure region, wherein the pressure reduction unit is configured as an additional flow resistance and for depressurising the fluid and conveying the fluid out of the enrichment apparatus.

39. The enrichment apparatus according to claim 38, wherein the enrichment apparatus is configured as a device for carrying out the method of claim 33.
